(19) United States
(12) Patent Application Publication
(10) Pub. No.: US 2021/0338609 A1
(43) Pub. Date: Nov. 4, 2021

(54) COMPOSITIONS FOR ADMINISTRATION TO ANIMALS TO INCREASE GUT NON-PROTEIN NITROGEN LEVELS

(71) Applicant: D.I.T Technologies Ltd, Toowoomba (AU)

(72) Inventors: Thomas Robinson Feez, Withcott (AU); Mark Rawlinson Peart, Geham (AU); Anthony David Wood, Corinda (AU)

(21) Appl. No.: 17/282,996

(22) PCT Filed: Oct. 4, 2019
(86) PCT No.: PCT/AU2019/051075

§ 371 Date: Apr. 5, 2021

(30) Foreign Application Priority Data
Oct. 5, 2018 (AU) 2018903768
Jan. 31, 2019 (AU) 2019900281

(51) Int. Cl. A61K 31/17; A61K 9/00; A61K 33/26; A61K 33/34; A61K 33/06; A61K 33/04; A61K 33/30; A61K 33/24; A61K 31/7004; A61P 3/02; A23K 20/105; A23K 20/20; A23K 20/24; A23K 20/163; A23K 50/15

(52) U.S. Cl. CPC A61K 31/17; A23K 50/15; A61K 33/26; A61K 33/34; A61K 33/06; A61K 33/04; A61K 33/30; A61K 33/24; A61K 31/7004; A61P 3/02; A23K 20/105; A23K 20/30; A23K 20/24; A23K 20/163; A61K 9/0053

(57) ABSTRACT

The present invention is concerned with increasing protein intake in a ruminant animal by supplementing the diet of the ruminant animal with urea phosphate. There can also be supplementation with a water soluble sulfur-containing salt such as ammonium sulfate. Urea can be administered together with urea phosphate. The composition provides a slow release form of urea that avoids inducing ammonia toxicity in the animal and is water soluble for administration to the animal in drinking water.

### TECHNICAL FIELD

The present invention relates to compositions for administration to animals to increase gut non-protein nitrogen levels. More particularly, the present invention relates to such compositions for administration to animals in drinking water.

### BACKGROUND

Any references to methods, apparatus or documents of the prior art are not to be taken as constituting any evidence or admission that they formed, or form part of the common general knowledge.

Grasses in northern Australia provide high quality grazing while they are actively growing, but as they mature they become more fibrous and difficult to digest, and so their feed value declines. This lowered nutritional value can cause weight loss in the livestock as the dry season progresses. Without intervention through an improvement in pasture quality, from rain or by way of appropriate supplementation, this downward spiral of declining feed intake of poorer quality feed can eventually result in animal deaths.

Urea is widely used as a feed supplement for ruminant livestock such as cattle, sheep and goats. Supplementation with urea increases the gut non-protein nitrogen levels in order to maintain healthy gut bacterial micro-flora. Increased growth of the rumen micro-flora leads to more effective fibre utilisation, and increased microbial protein production. These microbes are subsequently flushed out of the rumen and are digested lower down the digestive system. Therefore supplementation with urea provides a source of protein to the livestock animal. However, urea can and will kill cattle if consumed too quickly, and therefore must be administered carefully.

Urea supplements can be administered by way of dry lick blocks which are positioned in accessible positions in a field for the livestock animals to lick. The disadvantage with the use of such dry lick blocks is that livestock can overdose on the urea intake. This can result in toxicity to the animal because an enzyme present in the rumen, urease, produces ammonia when it catalyses the degradation of urea, and ammonia is toxic to ruminant animals in high blood concentration. Accordingly, ingredients such as salt and molasses must be added to dry lick recipes to reduce the risk of urea toxicity by modifying the palatability of the lick. In addition, access to the lick blocks can result in competition between the animals, which can result in less dominant animals having restricted or no access to the lick blocks. This, in turn, can result in lower animal yield as some animals will not have sufficient urea intake. Further, the urea blocks can be expensive to buy and deliver on site on so called “lick runs” (the cost is approximately AUD0.60 per day per block).

### SUMMARY OF INVENTION

The present invention relates to compositions with a slow release characteristic for administration to animals to increase gut non-protein nitrogen levels, and which are adapted to be dispensed into a livestock water supply. As a result, the present invention allows for more uniform supply of urea across each of the livestock animals. The present invention also provides improved safety due to the slow release characteristic of the composition. The slow release characteristic decreases the prospect that the livestock animal will suffer toxicity due to high ammonia blood concentration. It also obviates the need to supplement the urea with fillers such as molasses, cottonseed and grain, and further obviates the need to deliver dry urea blocks to the field.

In one aspect there is provided a physiologically acceptable composition for metering into a drinking water supply for a ruminant animal, said composition being an aqueous solution comprising a nutritionally effective amount of urea phosphate as an active ingredient, wherein the total active ingredient concentration of the composition is greater than 10% w/w.

In another aspect there is provided a physiologically acceptable composition for metering into a drinking water supply for a ruminant animal, said composition being an aqueous solution comprising as active ingredients a nutritionally effective amount of:
urea phosphate; and
a water soluble sulfur-containing salt;
   wherein the total active ingredient concentration of the composition is greater than 10% w/w.

In another aspect there is provided a physiologically acceptable composition for metering into a drinking water supply for a ruminant animal, said composition being an aqueous solution comprising as active ingredients a nutritionally effective amount of:
a. urea phosphate;
b. urea; and
c. a water soluble sulfur-containing salt;
   wherein the total active ingredient concentration of the composition is greater than 10% w/w.

In another aspect there is provided a physiologically acceptable composition for metering into a drinking water supply for a ruminant animal, said composition being an aqueous solution comprising as active ingredients a nutritionally effective amount of:
a. urea phosphate; and
b. urea;
   wherein the total active ingredient concentration of the composition is greater than 10% w/w.

In another aspect there is provided a physiologically acceptable composition, said composition being an aqueous solution comprising a nutritionally effective amount of urea phosphate as an active ingredient, wherein the total active ingredient concentration of the composition is greater than 10% w/w, when metered into a drinking water supply for a ruminant animal.

In another aspect there is provided a physiologically acceptable composition, said composition being an aqueous solution comprising as active ingredients a nutritionally effective amount of:
urea phosphate; and
a water soluble sulfur-containing salt;
   wherein the total active ingredient concentration of the composition is greater than 10% w/w, when metered into a drinking water supply for a ruminant animal.

In another aspect there is provided a physiologically acceptable composition, said composition being an aqueous solution comprising as active ingredients a nutritionally effective amount of:
a. urea phosphate;
b. urea; and
c. a water soluble sulfur-containing salt;
   wherein the total active ingredient concentration of the composition is greater than 10% w/w, when metered into a drinking water supply for a ruminant animal.

In another aspect there is provided a physiologically acceptable composition, said composition being an aqueous solution comprising as active ingredients a nutritionally effective amount of:
a. urea phosphate; and
b. urea;
   wherein the total active ingredient concentration of the composition is greater than 10% w/w, when metered into a drinking water supply for a ruminant animal.

In another aspect there is provided a method of administering urea to a ruminant animal with reduced risk of ammonia toxicity, comprising metering a composition as described herein into a drinking water supply for the ruminant animal.

In yet another aspect there is provided a method of increasing non-protein nitrogen intake in a ruminant animal comprising supplementing the diet of the ruminant animal by metering a composition as described herein into a drinking water supply for the ruminant animal.

In yet another aspect there is provided a method of increasing available protein in a ruminant animal comprising supplementing the diet of the ruminant animal by metering a composition as described herein into a drinking water supply for the ruminant animal.

In still another aspect there is provided a kit comprising a composition as described herein and instructions for metering the composition into a drinking water supply for the ruminant animal.

### DETAILED DESCRIPTION OF PREFERRED EMBODIMENTS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way.

Administration of urea phosphate to a ruminant animal in drinking water provides an increase in non-protein nitrogen intake in the animal. Therefore the present invention contemplates supplementing the diet of the ruminant animal with urea phosphate by administration of the urea phosphate in the drinking water of said ruminant animal. This, in turn, increases growth of the rumen micro-flora, which leads to more effective fibre utilisation and increased microbial protein production. Since the microbes are flushed out of the rumen in time, and digested lower down the digestive system of the animal, the increase in non-protein nitrogen ultimately increases the availability of protein to the livestock animal.

The urea phosphate provides for slow release of urea after ingestion by a livestock animal due to its relative stability to digestion by the urease enzyme. However, it is broken down slowly, and it provides a source of dietary nitrogen and phosphorus when broken down. The provision of urea in the form of urea phosphate provides the advantage of slow release nutritional benefits at the required release rates and doses.

The formulation of the present invention has unique properties that make it highly desirable in the formulation of water soluble supplements. While not wishing to be bound by theory, it is believed that urea toxicity occurs in ruminants when too much urea is broken down by urease into ammonia too quickly. If this happens the ammonia is rapidly adsorbed into the blood stream in levels which are high enough to be toxic. Urea phosphate is a molecule in which the urea molecule is bonded to the phosphoric acid molecule. This bond has to be split before urease can act on the urea molecule. The strength of the urea phosphate bond means that this break down takes time, thus making it a slow release and safe form of urea. As well as providing a slow release and safe form of urea, urea phosphate is strongly acidic (pH 2) and is used to buffer the high bicarbonate bore waters. This provides stabilisation of the urea and slows its decay to ammonia. Urea phosphate is a highly hygroscopic solute. The addition of urea phosphate in the formulation means that when thoroughly mixed and dissolved, the product will remain in complete liquid solution when dispensed into livestock drinking water. This prevents any product settling in troughs and throughout lengths of poly pipe. Urea phosphate also provides a rapidly soluble source of phosphorus, which is a vital element for the growth of cattle, sheep and goats. Unlike many other phosphorus supplements, when fed through a water supplementing system, the phosphorus contained in urea phosphate is 100% available to the ruminant.

As a matter of practicality, urea supplementation ordinarily takes place in conjunction with supplementation with sulfur. Sulfur constitutes around 10% of a protein molecule. Accordingly, in embodiments sulfur is provided along with nitrogen to ensure that sulfur is not a limiting factor in the production of bacterial protein. The aim of a urea supplementation program is to improve the rumen function and animal performance by supplying a small amount of urea and sulfur-containing compounds to cattle on at least a daily basis during the dry season

Accordingly, the invention provides for an increase in protein availability in a ruminant animal through supplementing the diet of the ruminant animal with urea phosphate and a water soluble sulfur-containing salt in the drinking water of the ruminant animal. Thus it also relates to a physiologically acceptable composition comprising:
urea phosphate; and
a water soluble sulfur-containing salt.

In an embodiment the water soluble sulfur-containing salt is a sulfate.

In an embodiment the water soluble sulfur-containing salt is selected from the group consisting of ammonium sulfate, zinc sulfate, cobalt sulfate, manganese sulfate, iron sulfate, copper sulfate and magnesium sulfate.

In an embodiment, the invention provides a physiologically acceptable composition comprising:
urea phosphate; and
ammonium sulfate

The urea phosphate provides for slow release of urea after ingestion by a livestock animal due to its relative stability to digestion by the urease enzyme. The composition of the present invention also provides for a source of dietary nitrogen, sulfur and phosphorus from the breakdown of urea and ammonium sulfate. The combination of urea phosphate and a water soluble source of sulfur, particularly ammonium sulfate, especially in the proportions as described herein, provides the advantage of slow release nutritional benefits at the required release rates and doses.

The present invention allows supplementation of the diet of a ruminant animal with urea, with reduced risk of ammonia toxicity. This can be done by administering urea phosphate or by administering urea in conjunction with urea phosphate. Accordingly, there is provided a method of administering urea to a ruminant animal with reduced risk of ammonia toxicity, comprising dispensing urea phosphate or urea in conjunction with urea phosphate into a drinking water supply for the animal.

In an embodiment, urea and urea phosphate are mixed prior to addition to the drinking water. In an embodiment urea is mixed into water and urea phosphate added in sufficient quantities to prevent rapid breakdown of urea. In an embodiment urea and urea phosphate are pre-mixed, and are supplied in a container which is adapted for direct connection to an apparatus for dispensing supplements into drinking water supply. In an embodiment the drinking water is from a highly alkaline source. In an embodiment, the water is bore water.

In an embodiment, urea and urea phosphate are metered into the drinking water. In an embodiment urea phosphate is metered into the drinking water in an amount suitable to prevent rapid breakdown of urea.

While not wishing to be bound by theory, the low pH of the urea phosphate solution reduces the pH in the rumen sufficiently that urease activity is reduced. Urease activity is strongly dependant on acidity. For example, it is strongest around pH 7. It is inhibited in acid environments. This means that animals being fed urea phosphate can tolerate being fed urea to a greater extent than usual. For example, where the rumen is around pH 5 an animal can tolerate 200 grams, or more in one feed without toxicity.

As used herein, the term “active ingredient”, or its equivalents, refers to substances that perform a role in enhancing the well-being of ruminant animals, as described herein. This may be by enhancing desirable process such increasing protein availability or by reducing adverse effects such toxicity.

An active ingredient is supplied in a nutritionally effective amount. As used herein, the term “nutritionally effective amount” refers to an amount that will be effective in enhancing desirable process in an animal, such as increasing protein availability or by reducing adverse effects such toxicity, when introduced in that amount in the drinking water. In the case of urea phosphate, a nutritionally effective amount is an amount in the drinking water that is sufficient to increase non-protein nitrogen intake in the animal, when ingested alone or when ingested in conjunction with other active ingredients, in particular, a water soluble sulfur-containing salt and/or urea.

It will be appreciated that introduction of active ingredients into the water supply means the amount ingested by the animal will depend upon water intake, and the concentration of active ingredients is calculated to ensure administration of an appropriate amount. The daily water requirements and intake by livestock varies considerably according to class of stock, production status, age and condition of the animal, dry matter intake, quality and nature of feed, climatic conditions, and the quality of the water. For example, while the average daily water intake for beef cattle is about 45 L, in northern Australia hot summer temperatures significantly increase daily intake of water. Lactating cows may have a 30% higher daily water intake than dry cows. Furthermore, the requirements for Bos taurus cattle in hot conditions will be higher than those of Bos indicus cattle. It will also be appreciated that administration of the active ingredients in very high amounts may not show enough benefit to justify the additional cost. Adjustments can be made in the concentration of active ingredients in the composition to be administered and/or in the rate of metering the composition so that the animal ingests an amount that is beneficial and cost effective.

The person skilled in the art will understand that a user can monitor the beneficial effect of the active ingredients by monitoring for signs such the weight of animals. In particular, they can compare the rate of weight gain (or reduction in weight loss in stressed animals) in animals treated with a composition described herein, and compare this to a baseline established for untreated animals.

As used herein, the term “total active ingredients”, or its equivalents, refers to the sum of the amount of the different active ingredients that may be present in the composition. A percentage of total active ingredients is a percentage by weight of the dry ingredients.

In an embodiment, the urea phosphate may be included at a rate of 10-100% w/w of the total active ingredients composition.

In an embodiment, the urea phosphate may be included at a rate of 15-50% w/w of the total active ingredients.

In an embodiment, the urea phosphate may be included at a rate of 20-35% w/w of the total active ingredients.

In an embodiment, the ammonium sulfate may be included at a rate of 0-20% w/w of the total active ingredients.

In an embodiment, the ammonium sulfate may be included at a rate of 3-15% w/w of the total active ingredients.

In an embodiment, the ammonium sulfate may be included at a rate of 5-10% w/w of the total active ingredients.

In an embodiment, the urea may be included at a rate of 0-55% w/w of the total active ingredients.

In an embodiment, the urea may be included at a rate of 20-55% w/w of the total active ingredients.

In an embodiment, the urea may be included at a rate of 30-55% w/w of the total active ingredients.

In an embodiment the composition comprises:
20-35% w/w of total active ingredient concentration of urea phosphate; and
5-10% w/w of the total active ingredients of ammonium sulfate.

In an embodiment the composition comprises:
20-35% w/w of the total active ingredients of urea phosphate;
30-55% w/w of the total active ingredients of urea; and
5-10% w/w of the total active ingredients of ammonium sulfate.

In an embodiment, the dry active ingredients are blended before dissolution in water. Alternatively, the desired amount of each active ingredient is mixed into the solvent to form a solution of the desired concentration. In this way, the physiologically acceptable composition is formulated as a concentrate for application into the water supply of ruminant animals.

In an embodiment, the physiologically acceptable composition may have a pH of substantially 2. In this way, the composition acts as a buffer to alkaline water sources high in bicarbonate to stabilise the urea and urea phosphate to prevent its early decay to ammonia and aid in its slow release property. The capacity of a composition containing urea to produce ammonia toxicity in ruminant animals can be measured with a urease test strip. These strips contain the enzyme urease. Urease is specific to urea and catalyses the breakdown to ammonia, and the strip undergoes a colour change when the urease strip is exposed to ammonia. While urea phosphate does break down to urea this occurs slowly, so ammonia is generally not detectable in solutions containing urea phosphate.

Urease activity is reduced in an acid environment. The pH change induced by addition of urea phosphate reduces urease activity so the amount of urea, if it is present, converted by the enzyme is greatly reduced. This means that urea can be added to the formulation, which is advantageous since urea is a much cheaper source of nitrogen than urea phosphate.

In an embodiment, if a user desires to test for ammonia production, for example if urea is used in relatively high amounts and a relatively alkaline water source such as bore water is used, a urease test strip can be provided.

Certain plant based chemicals can cause toxicity to ruminant animals such as cattle or sheep if ingested. For example, sorghum grown in stressed situations can have increased levels of cyanogenic glycosides. If ingested by ruminant animals this can be is converted into prussic acid. The levels are normally higher in plants that have suffered through drought, and even more likely to be elevated once a drought has broken and plants begin to grow. Cattle suffering from prussic acid poisoning display symptoms including difficulties in breathing, anxiety, muscle tremors, bloat and convulsions. Once the toxin is ingested the poisoning occurs quite quickly, and animals can be dead within minutes.

In an embodiment the composition is designed to reduce the effects of prussic acid on ruminant livestock animals such as cattle and sheep grazing forage sorghum. The formulation is also ideal for cattle grazing Leucaena and for sheep and cattle grazing mulga country. In an embodiment the formulation of the present invention contains a source of sulfur, generally in the form of a water soluble salt. In an embodiment the water soluble salt is a sulfate. In an embodiment the water soluble sulfate is selected from the group consisting of lithium sulfate, sodium sulfate, potassium sulfate, ammonium sulfate, magnesium sulfate, zinc sulfate and copper sulfate. In an embodiment the water soluble sulfate salt is magnesium sulfate. The introduction of a source of sulfur is believed to increase the animal's efficiency to convert prussic acid into a non-toxic form, reducing their likelihood of being poisoned.

Another example of a plant based chemical which can decrease yield of ruminant livestock animals are tannins which are contained in the Mulga (Acacia aneura) tree native to regional areas of Australia. Tannins are a biomolecule, which bind to protein in the plant. This binding prevents ruminant animals from accessing all of the available protein when grazing on mulga resulting in a loss of production for graziers.

By adding a source of soluble PEG, in an embodiment, the formulation is able to offer graziers specifically in the mulga lands a product designed to maximise productivity. Polyethylene glycol (PEG) is a compound that binds the tannins, freeing up the amount of available protein. Sheep supplemented with polyethylene glycol (PEG) showed a significant response in feed intake, live weight increase and increased wool fibre diameter.

In another embodiment the composition further comprises a source of sulfur other than the water soluble sulfur-containing salt. In an embodiment, the source of sulfur is present in an amount of between 1 and 5% w/w of the total active ingredients. In an embodiment, the amount of the sulfur source may be between 2 and 4% w/w of the total active ingredients. In an embodiment, the amount of the source of sulfur may be between 2 and 4% w/w of the total active ingredients. In an embodiment, the amount of the source of sulfur may be between 2.5 and 3.5% w/w of the total active ingredients. In an embodiment, the amount of the source of sulfur may be 2.4% w/w of the total active ingredients.

In an embodiment, the physiologically acceptable composition may also comprise an amount of polyethylene glycol. In an embodiment, the amount of polyethylene glycol may be between 1 and 10% w/w of the total active ingredients. In an embodiment, the amount of polyethylene glycol may be between 3 and 7% w/w of the total active ingredients. In an embodiment, the amount of polyethylene glycol may be between 4 and 6% w/w of the total active ingredients. In an embodiment, the amount of polyethylene glycol may be 5% w/w of the total active ingredients.

In another embodiment of the present invention there broadly resides a composition comprising the following active agents: a. 20-35% w/w of the total active ingredients of urea phosphate; b. 5-10% w/w of the total active ingredients of ammonium sulfate; and c. 5-10% w/w of the total active ingredients of a source of sulfur, wherein the composition is water soluble for administration in drinking water to a ruminant animal.

In an embodiment, the composition further comprises between 1 and 10% w/w of the total active ingredients of polyethylene glycol.

A further embodiment provides a composition comprising the following active agents: a. 20-35% w/w of the total active ingredients of urea phosphate; b. 30-55% w/w of the total active ingredients of urea; and c. 5-10% w/w of the total active ingredients of ammonium sulfate d. 5-10% w/w of the total active ingredients of magnesium sulfate, wherein the composition is water soluble for administration in drinking water to a ruminant animal.

In an embodiment, the composition further comprises between 1 and 10% w/w of the total active ingredients of polyethylene glycol.

In an embodiment the composition further comprises trace elements. In general trace elements are included at supplement levels to ensure that adequate quantities are available. When administered in conjunction with a carbohydrate capable of acting as an energy source and/or carbon source for amylose degrading bacteria, such as dextrose, glucose or fructose, and a source of magnesium, trace elements are effective in stimulating cellulose digestion in the rumen, increasing net available energy and a reducing physiological stress in the animal to which the trace elements are administered. This, in turn, leads to greater disease resistance and calmer animals as well as a greater utilisation of the available diet, or creates an energy sparing effect for existing diets. However, while not wishing to be bound by theory, it is believed that certain trace elements perform a secondary role in slowing the release of ammonia from urea by inhibition of the enzyme urease. In an embodiment the trace elements are selected from the group consisting of magnesium, zinc, copper, cobalt and manganese.

The physiologically acceptable composition is formulated as a concentrate for dispensation into the water supply of ruminant animals. The concentrate can be administered by adding a measured amount to a source of drinking water such as a drinking trough. Advantageously the concentrate is metered into a drinking water supply. In particular, it may be proportionally dosed through the Nutridose or NutriPro dosing units (Direct Injection Technologies).

It is advantageous for the composition to be provided as a concentrated solution. Typically the composition is provided in a container. Transport costs are minimised by transporting the least amount of water, hence it is advantageous for the composition to be concentrated. However, provision of a highly concentrated composition would generally require that the user dilute the composition. It has now been found that a concentrated composition can be metered into the drinking water of a ruminant animal through a dosing unit such as the Nutridose or NutriPro dosing units (Direct Injection Technologies). Accordingly, in an embodiment the composition is metered into the drinking water of the ruminant animal directly from the container in which it is transported.

As used herein, the term “metered” or its equivalents refers to a measured dispensation of a composition as described herein into a drinking water supply. The rate of dispensation is monitored and controlled to ensure that a desired concentration of the composition in the drinking water is achieved. This, in turn, ensures that a nutritionally effective amount of the active ingredients contained in the composition is delivered to animals drinking from the water supply. The rate of dispensation may be adjusted periodically to maintain the concentration of active ingredients in the drinking water supply if conditions change, or to adjust the concentration of active ingredients in the drinking water supply.

As used herein, the term “concentration”, or its equivalents, refers to the concentration of active ingredients in the composition. Unless otherwise specified, the concentration will be the concentration of total active ingredients. The concentration will be expressed herein as the mass of total active ingredients per unit mass of the composition.

Generally, the urea phosphate solution has a concentration of greater than 10% w/w.

In an embodiment the concentration of the composition is up to 60% w/w.

In an embodiment the concentration of the composition is from 20 to 60% w/w.

In an embodiment the concentration of the composition is from 40 to 60% w/w.

In an embodiment the concentration of the composition is about 60% w/w.

In an embodiment the composition is metered to provide a final concentration of from 0.1 to 5.0 g per litre of water before consumption by at least one ruminant animal.

In an embodiment the composition is metered to provide a final concentration of from 0.5 to 2.0 g per litre of water before consumption by at least one ruminant animal.

In an embodiment the composition is metered to provide a final concentration of from 0.8 to 1.2 g per litre of water before consumption by at least one ruminant animal.

In an embodiment, the composition is metered to provide a final concentration of substantially 1 g per litre of water before consumption by at least one ruminant animal.

In one aspect there is provided a kit comprising a composition as described herein and instructions for administration in drinking water to a ruminant animal.

In an embodiment the kit further comprises a urease test strip.

### EXAMPLES

### Example 1

The composition was manufactured as a dry concentrate by mixing in a mixer the following ingredients:
35% urea phosphate;
55% urea; and
10% ammonium sulfate.

The dry mix concentrate was dissolved in water to form a liquid concentrate at a concentration of 600 kg of the dry mix concentrate in approximately 400 litres of water to achieve the below liquid formulation:
22% urea phosphate;
33% urea; and
5% ammonium sulfate.
   Additional nutrient ingredients can be added during manufacture without departing from the scope of the present invention.

### Example 2

The 60% w/w liquid concentrate can be direct injected into the livestock water supply via a peristaltic pump using NutriPro dosing units (Direct Injection Technologies).

A paddock of approximately 2000 older cows with calves located close to the Queensland border on the Barkly tableland were used to determine the effectiveness of water supplementation as opposed to dry lick. The factors used to determine this were weight gain, dry feed consumption and conception rate. The cattle were consistently fed a 60% solution of urea, urea phosphate and ammonium sulfate as described in Example 1 through a Nutridose unit (Direct Injection Technologies) in their drinking water supply. The cattle were on supplemented water inconsistently for a period of 7 months. Thereafter, they the cattle were consistently on the supplemented water for a period of 4 months, with the rate of administration of the supplement being on average 1 gram per litre of drinking water. A control mob was supplemented with traditional dry lick.

At the end of the 4 months of consistent treatment, the treated mob were on average 20 kg heavier than the control mob. The treated mob showed half a body score index improvement compared to the control mob. Furthermore, the treated mob produced a 20% increase in conception rate, achieving 35%.

### Example 3

The slow release characteristic of urea phosphate was demonstrated in a simple test using an Azostix® reagent test strip (Siemens) designed to measure the level of blood urea nitrogen. These strips contain the enzyme urease and the indicator bromothymol blue. Urease is specific to urea and breaks it down to ammonia. It only takes 60 seconds to do this. Therefore, the strip turns from yellow to green when the urease strip is exposed to urea. One strip was exposed to a solution of urea at the rate of 1 gram per litre. As expected, this strip turned from the unexposed yellow colour to a deep green, demonstrating ammonia production.

A second strip was exposed to urea phosphate at 5 grams per litre. The urea phosphate strip was unchanged. The urease has no effect in converting urea phosphate to ammonia.

A third strip was exposed to the formulation from Example 1 at the rate of 2 grams per litre (1.5 grams per litre urea). The strip turned slightly green, showing a slow rate of conversion of this formula. This shows a much slower rate of conversion of urea to ammonia even though the urea concentration is higher than the strip exposed to urea alone. This demonstrates that breakdown of urea is greatly reduced in the presence of urea phosphate due to the low pH.

In terms of the speed of breakdown of urea to ammonia, all of the urea in the urea only mix was converted to ammonia within 60 seconds. For the urea phosphate none of the urea was converted as the urea was not available to the urease enzyme and the enzyme action was reduced in the acid environment. For the formulation of Example 1, less than 1% of the urea was converted to ammonia within the 60 seconds. This demonstrates the slow release function of the formula, improving safety by slowing the release of urea to ammonia.

### Example 4

A composition was manufactured as a dry concentrate by mixing in a mixer the following ingredients: 35% urea phosphate; 55% urea; 10% ammonium sulfate and 5% magnesium sulfate. The dry mix concentrate was either packaged into 25 kg sacks for transport to distributers or 600 kg of the dry mix concentrate dissolved in 400 litres of water to achieve the below liquid formulation: 22% urea phosphate; 33% urea; 5% ammonium sulfate; and 2.4% magnesium sulfate. The formulation is available in a dry bagged form to be mixed with water in a mixing tank or as a liquid product designed to be fed into a livestock water source through a dispensing system as described in Example 2.

Therefore, at least in embodiments, the present invention provides a number of advantages over the prior art such as:
improved ease of use due to high solubility and hygroscopy which allows dispensing of the composition of the present invention into a livestock water supply and more uniform urea supply across each of the livestock animals via the water intake proportionate to each animal's body weight;
improved safety due to the slow release characteristic of the composition of the present invention (due to chemical composition and high acidity) to decrease toxicity to livestock animal's due to reduced ammonia build up;
improved cost saving to the user by obviating the need to supplement the urea with fillers such as molasses, cottonseed, grain etc and the need to deliver dry urea blocks to the field; and
direct dispensing of a liquid concentrate into a livestock water supply to avoid any need for handling or mixing.

In compliance with the statute, the invention has been described in language more or less specific to structural or methodical features. The term “comprises” and its variations, such as “comprising” and “comprised of” is used throughout in an inclusive sense and not to the exclusion of any additional features.

It is to be understood that the invention is not limited to specific features shown or described since the means herein described comprises preferred forms of putting the invention into effect.

The invention is, therefore, claimed in any of its forms or modifications within the proper scope of the appended claims appropriately interpreted by those skilled in the art.

1. A method of increasing protein availability in a ruminant animal comprising supplementing the diet of the ruminant animal by metering a physiologically acceptable composition into the drinking water of the ruminant animal said composition being an aqueous solution comprising a nutritionally effective amount of urea phosphate as an active ingredient, wherein the total active ingredient concentration of the composition is greater than 10% w/w and wherein the urea phosphate is included at a rate of 20-35% w/w of the total active ingredients.

2. (canceled)

3. The method as claimed in claim 1, wherein the physiologically acceptable composition further comprises as an active ingredient a nutritionally effective amount of a water soluble sulfate.

4. (canceled)

5. The method as claimed in claim 3 wherein the water soluble sulfate is selected from the group consisting of ammonium sulfate, iron sulfate, copper sulfate and magnesium sulfate.

6. (canceled)

7. The method as claimed in claim 3 wherein the water soluble sulfate is included at a rate of 5-10% w/w of the total active ingredients.

8. The method as claimed in claim 1, wherein the physiologically acceptable composition further comprises a nutritionally effective amount of urea as an active ingredient.

9. The method as claimed in claim 8, wherein the urea is included at a rate of 30-55% w/w of the total active ingredients.

10. The method as claimed in claim 1 wherein the physiologically acceptable composition has a pH of substantially 2.

11. (canceled)

12. (canceled)

13. (canceled)

14. (canceled)

15. The method as claimed in claim 1, wherein the physiologically acceptable composition further comprises a nutritionally effective amount of trace elements that inhibit urease as an active ingredient.

16. The method as claimed in claim 15, wherein the trace elements are selected from the group consisting of magnesium, zinc, copper, cobalt and manganese.

17. The method as claimed in claim 1, wherein the physiologically acceptable composition further comprises a nutritionally effective amount of carbohydrate capable of acting as an energy source and/or carbon source for amylase degrading bacteria.

18. The method as claimed in claim 17, wherein the carbohydrate is selected from the group consisting of dextrose, glucose and fructose.

19. (canceled)

20. The method as claimed in claim 1, wherein the total active ingredient concentration of the composition is from 20 to 60% w/w.

21. (canceled)

22. (canceled)

23. (canceled)

24. A method as claimed in claim 1, wherein the drinking water is from a highly alkaline source.

25. A method as claimed in claim 24, wherein the water is bore water.

26. (canceled)

27. (canceled)

28. A method as claimed in claim 1, wherein the physiologically acceptable composition is metered to provide a final concentration of from 0.1 to 5.0 g per litre of water before consumption by at least one ruminant animal.

29. A method as claimed in claim 28, wherein the physiologically acceptable composition is metered to provide a final concentration of from 0.5 to 2.0 g per litre of water before consumption by at least one ruminant animal.

30. A method as claimed in claim 28, wherein the physiologically acceptable composition is metered to provide a final concentration of from 0.8 to 1.2 g per litre of water before consumption by at least one ruminant animal.

31. A method as claimed in claim 28, wherein the physiologically acceptable composition is metered to provide a final concentration of substantially 1 g per litre of water before consumption by at least one ruminant animal.

32. A method as claimed in claim 1, wherein the physiologically acceptable composition is metered directly from the container in which the composition is provided.

33. (canceled)

34. (canceled)

35. (canceled)
